SEQUENCE LISTING (2 sequences)

SEQ ID NO: 1 - DNA, 1464 bp, Homo sapiens
atgagcctccccaattcctcctgcctcttagaagacaagatgtgtgagggcaacaagaccactatggccagcccccagctgatgcccctggtggtggtcctgagcactatctgcttggtcacagtagggctcaacctgctggtgctgtatgccgtacggagtgagcggaagctccacactgtggggaacctgtacatcgtcagcctctcggtggcggacttgatcgtgggtgccgtcgtcatgcctatgaacatcctctacctgctcatgtccaagtggtcactgggccgtcctctctgcctcttttggctttccatggactatgtggccagcacagcgtccattttcagtgtcttcatcctgtgcattgatcgctaccgctctgtccagcagcccctcaggtaccttaagtatcgtaccaagacccgagcctcggccaccattctgggggcctggtttctctcttttctgtgggttattcccattctaggctggaatcacttcatgcagcagacctcggtgcgccgagaggacaagtgtgagacagacttctatgatgtcacctggttcaaggtcatgactgccatcatcaacttctacctgcccaccttgctcatgctctggttctatgccaagatctacaaggccgtacgacaacactgccagcaccgggagctcatcaataggtccctcccttccttctcagaaattaagctgaggccagagaaccccaagggggatgccaagaaaccagggaaggagtctccctgggaggttctgaaaaggaagccaaaagatgctggtggtggatctgtcttgaagtcaccatcccaaacccccaaggagatgaaatccccagttgtcttcagccaagaggatgatagagaagtagacaaactctactgctttccacttgatattgtgcacatgcaggctgcggcagaggggagtagcagggactatgtagccgtcaaccggagccatggccagctcaagacagatgagcagggcctgaacacacatggggccagcgagatatcagaggatcagatgttaggtgatagccaatccttctctcgaacggactcagataccaccacagagacagcaccaggcaaaggcaaattgaggagtgggtctaacacaggcctggattacatcaagtttacttggaagaggctccgctcgcattcaagacagtatgtatctgggttgcacatgaaccgcgaaaggaaggccgccaaacagttgggttttatcatggcagccttcatcctctgctggatcccttatttcatcttcttcatggtcattgccttctgcaagaactgttgcaatgaacatttgcacatgttcaccatctggctgggctacatcaactccacactgaaccccctcatctaccccttgtgcaatgagaacttcaagaagacattcaagagaattctgcatattcgctcctaa

SEQ ID NO: 2 - DNA, 1080 bp, Homo sapiens
atggcacccaatggcacagcctcttccttttgcctggactctaccgcatgcaagatcaccatcaccgtggtccttgcggtcctcatcctcatcaccgttgctggcaatgtggtcgtctgtctggccgtgggcttgaaccgccggctccgcaacctgaccaattgtttcatcgtgtccttggctatcactgacctgctcctcggcctcctggtgctgcccttctctgccatctaccagctgtcctgcaagtggagctttggcaaggtcttctgcaatatctacaccagcctggatgtgatgctctgcacagcctccattcttaacctcttcatgatcagcctcgaccggtactgcgctgtcatggacccactgcggtaccctgtgctggtcaccccagttcgggtcgccatctctctggtcttaatttgggtcatctccattaccctgtcctttctgtctatccacctggggtggaacagcaggaacgagaccagcaagggcaatcataccacctctaagtgcaaagtccaggtcaatgaagtgtacgggctggtggatgggctggtcaccttctacctcccgctactgatcatgtgcatcacctactaccgcatcttcaaggtcgcccgggatcaggccaagaggatcaatcacattagctcctggaaggcagccaccatcagggagcacaaagccacagtgacactggccgccgtcatgggggccttcatcatctgctggtttccctacttcaccgcgtttgtgtaccgtgggctgagaggggatgatgccatcaatgaggtgttagaagccatcgttctgtggctgggctatgccaactcagccctgaaccccatcctgtatgctgcgctgaacagagacttccgcaccgggtaccaacagctcttctgctgcaggctggccaaccgcaactcccacaaaacttctctgaggtccaacgcctctcagctgtccaggacccaaagccgagaacccaggcaacaggaagagaaacccctgaagctccaggtgtggagtgggacagaagtcacggccccccagggagccacagacaggtaa

(19) United States
(12) Patent Application Publication
(10) Pub. No.: US 2004/0253675 A1
(43) Pub. Date: Dec. 16, 2004

(54) Method for GPCR assay with a coexpressed Galpha protein

(75) Inventors: Tomoko Takeshita, Kawagoe (JP); Jun Otomo, Tokyo (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/774,613

(22) Filed: Feb. 10, 2004

(30) Foreign Application Priority Data
Jun. 13, 2003 (JP) 2003-170032

(51) Int. Cl. C12P 21/02; C12N 5/06; C07K 14/705; C12N 15/85

(52) U.S. Cl. 435/69.1; 435/455; 435/320.1; 435/325; 530/350; 536/23.5

(57) ABSTRACT

This invention provides a method for assaying activities of signal transduction that enables identification of a ligand with a single assay method, thereby simplifying and accelerating the assay method for identifying a ligand of a GPCR with unknown functions. In this method, RNA encoding a GPCR and RNA encoding a chimeric Gqα subunit constituted by a portion of a G₁₁ or Gq subunit and a portion of a G₁₄, G₁₅, or G₁₆ subunit are transfected together to an oocyte removed from a Xenopus and selected by a conventional technique. After transfection of the RNAs, a ligand candidate substance is added to the oocyte that was cultured for a given period of time, and the activity is then assayed.

### TECHNICAL FIELD

The present invention relates to a method for assaying activity of signal transduction mediated by G-protein coupled receptors (hereafter referred to as “GPCR(s)”) utilizing animal cells such as Xenopus oocytes.

### BACKGROUND ART

Some of the GTP-binding proteins that specifically bind to a nucleotide GDP or GTP are referred to as G-proteins. Such G-proteins amplify and traduce signals into cells by coupling to GPCR on a cell membrane when an extracellular informational substance (agonists for GPCR) binds to the GPCR. A G-protein that is activated upon the binding of an agonist to a GPCR forms a trimer consisting of α, β, and γ subunits, thus, it is also referred to as a trimeric G-protein. There are a variety of G-protein subtypes that differ based on functions or other factors, such as Gs, Gi, Go, Gq, Gt, and Golf subtypes. For example, a G-protein that is activated by rhodopsin, which is one type of GPCR, is sometimes referred to as Gt or transducin. The Gq subtype is known to be further classified into, for example, Gq and G₁₁ to G₁₆ types, although the terms vary depending on animal species. In order to examine the functions of the G-protein α subunit, attempts have been made to prepare chimera proteins among the G₁₁ to G₁₆ subtypes (e.g., “Journal of Biochemistry,” Koji Nakamura et al., 1996, vol. 120, pp. 996-1001). In contrast, examples of known GPCRs include H₁ and H₂ receptors, M₁ to M₅ receptors, δ opioid receptors, mGlu1 receptors, and rhodopsin.

In the past, activity of signal transduction mediated by GPCRs used to be assayed utilizing expression systems for cultured animal cells such as Xenopus oocytes. In such cases, however, assay methods had to be altered in accordance with the G-protein subtype that would couple with a GPCR. In the assay system utilizing the Xenopus oocyte, for example, the GPCR that couples with the Gq subtype G-protein can be assayed by employing changes in Ca-dependent Cl current as an indicator. Concerning the Gi subtype G-protein, assay must be conducted by employing intracellular cAMP-dependent K channel activity as an indicator.

When the G-protein subtype in a target signal transduction system is known, a suitable assay method can be adopted in accordance with the subtype as mentioned above. With the progress in recent human genome projects, many proteins that are deduceded to be GPCRs based on nucleotide or amino acid sequence information have been discovered. In order to verify these functions, it is necessary to inspect whether or not a ligand candidate substance activates the signal transduction system by a protein of interest, which is deduced to be a GPCR. In this case, a G-protein subtype in the subject system is generally unknown based only on sequence information. Thus, verification of the activity by conventional techniques was time-consuming due to the necessity of employing several assay methods relating to a single ligand. In other words, even in the case of an agonist bound to a GPCR, activation could not be detected by an assay method if it was unsuitable. This disadvantageously results in the provision of false-negative judgments.

### SUMMARY OF THE INVENTION

The present invention provides a method for allowing a GPCR to be coexpressed with a specific G-protein in animal cells such as Xenopus oocytes. This method has been achieved as a result of concentrated studies in order to develop a single method for assaying the activation of the signal transduction system mediated by a GPCR even when the G-protein subtype that naturally couples with the GPCR is unknown.

More specifically, the present invention provides a method for preparing foreign protein-expressing cells, wherein genes encoding G-protein coupled receptors (GPCRs) and genes encoding the chimeric Gqα subunit, which is a chimeric protein constituted by a portion of a Gqα or G₁₁α subunit and a portion of a G₁₄α, G₁₅α, or G₁₆α subunit (all of these subunits are a subunits of Gq subtype G protein), are transfected into animal oocytes.

Types of GPCR employed in the present invention are not particularly limited. Examples thereof include H₁ and H₂ receptors, M₁ to M₅ receptors, δ opioid receptors, and mGlu1 receptors. Genes encoding GPCRs can be chemically synthesized by a conventional technique based on the nucleotide sequence information obtained from the database of GenBank or other institutions. The GPCR-coding gene may be RNA or DNA, and it can be adequately modified in a manner common in the art in order to enhance translation efficiency in the host to which the gene is transfected.

A gene encoding the Gqα subunit to be coexpressed with a GPCR, which may be DNA or RNA, can also be chemically synthesized in the manner as described above. In the present invention, the chimeric Gqα subunit must be a chimera protein constituted by a Gqα or G₁₁α subunit and a G₁₄α, G₁₅α, or G₁₆α subunit. The constitution of the chimeric Gqα subunit is not particularly limited, but for example, the sequence of the N-terminal side of the chimeric subunit is preferably derived from a Gq or G₁₁ subunit and the the sequence of C-terminal side thereof is preferably derived from a G₁₄, G₁₅, or G₁₆ subunit. An example of the constitution of the chimeric Gqα subunit constituted by a portion of the G₁₁α subunit and a portion of the G₁₄α subunit is shown in FIG. 1. A Gα subunit is known to comprise approximately three hundred and several dozen amino acids (full-length) and to comprise at its N-terminus the βγ subunit activation site and at its C terminus a receptor binding site.

Examples of animal cells in which the aforementioned gene is transfected to allow the foreign protein to be expressed include, but are not particularly limited to, established cell lines, insect cells, and oocytes of Xenopus or the like. Methods for allowing animal cells to express a GPCR, inspecting the functions, and searching for ligands or the like are common in the art, and there are many texts concerning such subjects (for example Saisentan Souyaku (State-of-the-art drug discovery), T. Nagao et al. (ed.), Kyoritsu Shuppan Co., Ltd., pp. 821-826, “Ohfan Juyoutai (Orphan receptor)”; Sigunaru Dentatsu Jikken-hou (Experiment on signal transduction), S. Ui (ed.), Yodosha Co., Ltd., Chapter 3, pp. 54-73, “Juyoutai Bunsi no Kaiseki (Analysis of receptor molecules).”) From the viewpoints of availability, handleability, and the like, use of Xenopus oocytes is particularly preferable.

Gene transfection into animal cells can be suitably conducted by a technique common in the art. Automatic or manual microinjection using a needle for sample transfection is reliable and preferable. The transfections of a gene encoding a GPCR and a gene encoding a chimeric Gqα subunit may be simultaneously carried out. Alternatively, a gene encoding a GPCR is first transfected and a gene encoding the chimeric Gqα subunit is then transfected 12 to 36 hours thereafter. This is particularly preferable because of the existence of an enhanced response.

The ratio of the amount of genes encoding the chimeric Gqα subunit to that of the genes encoding a GPCR is preferably 1:0.1 to 1:10. In the case of Xenopus oocytes, the amount of the genes encoding a GPCR is set in the range of 1 to 10 ng, and the amount of the genes encoding the chimeric Gqα subunit is set in the range of 1 to 10 ng. This can provide preferable results.

After the GPCR genes are transfected, culture is conducted for 1 to 3 days. Thus, oocytes in which the aforementioned two types of foreign proteins are expressed can be obtained.

The thus obtained foreign protein-expressing oocytes are used to assay the activation of the PI turnover. Thus, activation of the signal transduction system mediated by a GPCR caused upon ligand binding can be assayed. FIG. 2 schematically shows the PI turnover. An oocyte in which a GPCR and the chimeric Gqα subunit (“Gx” in the drawing) have been expressed is stimulated with a ligand. When the ligand is bound to the receptor, a trimeric G-protein is dissociated into a Gα subunit and a GβGγ dimeric protein. When activation of an enzyme phospholipase C (PLC) is induced by this dissociation, and it then degrades PIP2, which is a phospholipid on the cell membrane. As a result of this reaction, InsP3 is generated. InsP3 is bound to InsP3 in the rough endoplasmic reticulum (ER) in the cell and mobilizes Ca in the cell. As a result, the intracellular Ca concentration is elevated, which makes the intracellular Ca-dependent Cl channel open.

Any assay method can be employed as long as it can assay the activation of the Gq protein. Examples of assay methods that can be used include assay of the intracellular Ca concentration by a fluorescence method and a method of utilizing changes in Ca-dependent Cl current as indicators of intracellular Ca concentration. According to the present invention, whether a test substance is a ligand or not can be determined by a single assay technique regardless of the type of GPCR to which the ligand binds. FIG. 3 schematically shows the assay technique according to the present invention. RNA encoding a GPCR and RNA encoding a chimeric Gqα subunit are synthesized in vitro, and the resultants are transfected into the oocyte by microinjection. Upon the ligand binding to the GPCR, a signal transduction system is activated by the GPCR. As a result, the Ca-dependent Cl ion channel is opened as described above, a chlorine ion is released, and potential differences are generated between inside and outside the cells (response). This response can be detected using a microelectrode, thereby assaying the occurrence of the response to a ligand, i.e., the activation upon ligand binding.

At the time of assay, a test substance, which is a candidate of a ligand, is brought into contact with the aforementioned foreign protein-expressing oocyte. For example, whether or not Ca-dependent Cl response is generated as a result thereof is detected. In such a case, the use only of an oocyte in which a specific GPCR has been expressed is sufficient. Alternatively, the foreign protein-expressing oocytes can be prepared for several types of GPCRs to conduct several assays in parallel.

The present invention also provides a method for selling or assigning the foreign protein-expressing oocyte according to the present invention that allows specific GPCRs to be expressed. In this case, only the oocyte in which a specific type of GPCR is expressed may be sold or assigned. Alternatively, cells are prepared for several types of GPCRs, and they may be combined as a set, thereby selling or assigning the set. In such a case, information such as the type of the transfected gene or the date of transfection may be recorded on a label, and this label may be attached to the package of the cells.

The present invention also provides a method for selling or assigning a gene encoding a chimeric Gqα subunit constituted by a portion of a Gqα or G₁₁α subunit and a portion of a G₁₄α, G₁₅α, or G₁₆α subunit. A chimeric Gqα subunit is not particularly limited, however, the sequence of the N-terminal side thereof is preferably derived from a Gq or G₁₁ subunit and the sequence of the C-terminal side thereof is preferably derived from a G₁₄, G₁₅, or G₁₆ subunit. This gene is transfected into an animal cell together with a gene encoding a GPCR as described above. Thus, cells in which these genes are allowed to express can be prepared, and the resulting cells can be used for assaying signal transduction mediated by a GPCR activated upon ligand binding.

The present invention also provides a method for screening for a ligand by bringing various test substances in contact with the aforementioned foreign protein-expressing cells as mentioned above.

Screening can be conducted using the aforementioned sold or assigned foreign protein-expressing cells or using the foreign protein-expressing cells prepared using the aforementioned sold or assigned gene.

Alternatively, the screening method according to the present invention can also be provided as a service for screening for a ligand of a GPCR, wherein the foreign protein-expressing cells are prepared in response to a client's request, a ligand of the GPCR that is expressed in the aforementioned cells is screened for, and the obtained analysis data can be provided to the client.

The screening method according to the present invention enables simplification and acceleration of the assay method for identifying a ligand of a GPCR with unknown functions.

### BRIEF DESCRIPTION OF THE DRAWINGS

FIG. 1 shows an example of the chimeric Gqα subunit that is used in the present invention.

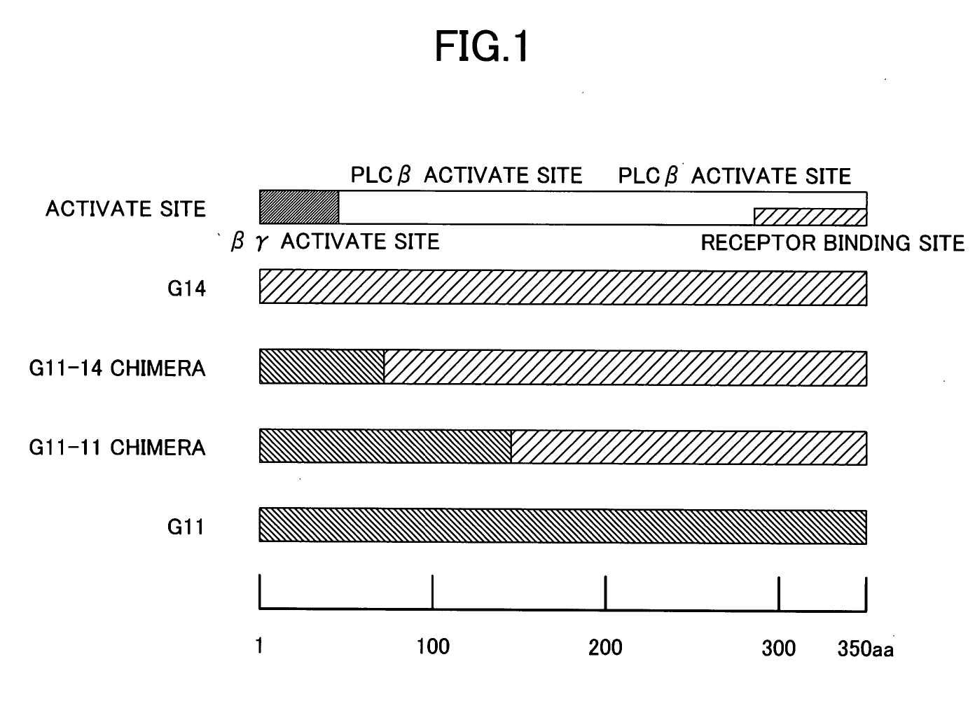

FIG. 2 schematically shows the PI turnover.

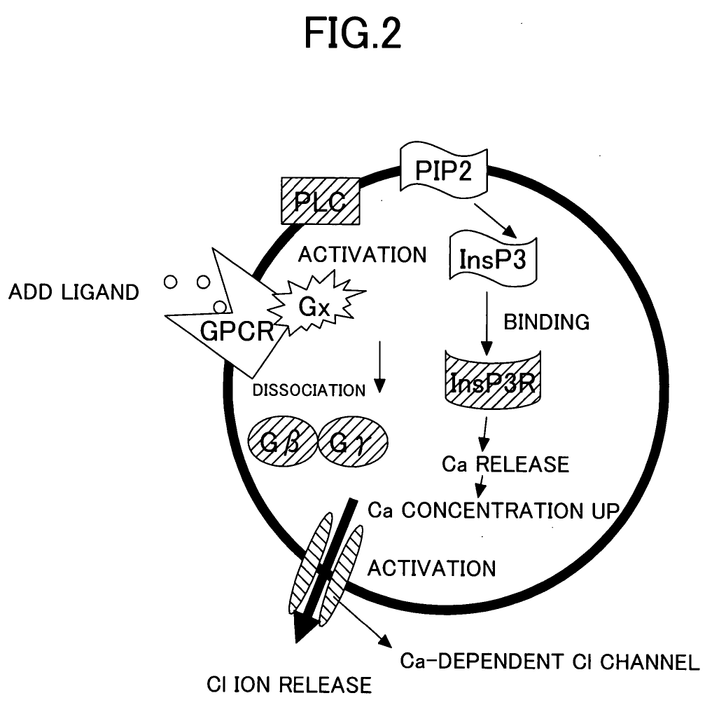

FIG. 3 schematically shows a process of preparing the foreign protein-expressing oocytes according to the present invention and a method for screening for ligands using the resulting oocyte.

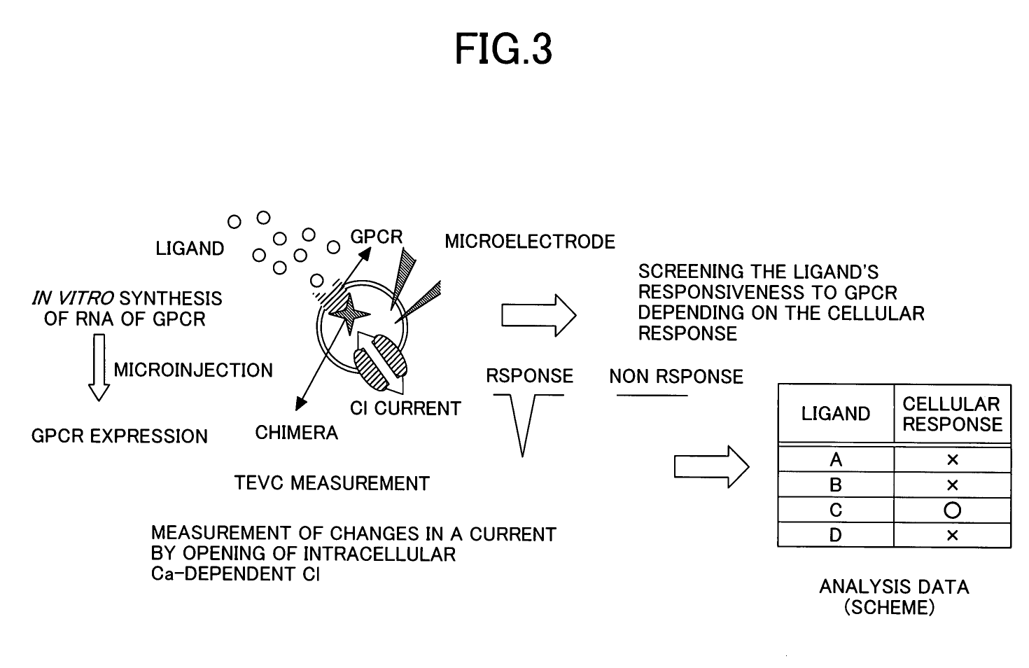

FIG. 4 shows the influence of coexpression of the chimeric Gqα subunit on the occurrence of Ca-dependent Cl response by ligand stimulation.

FIG. 5 shows the influence of the ligand concentration on the response in the oocyte coexpressing the chimeric Gqα subunit and a GPCR

FIG. 6 shows examples of responses in the oocyte to which the gene encoding the chimeric Gqα subunit was transfected simultaneously with the gene encoding a GPCR and in the oocyte to which the former gene was first transfected and the latter gene was then transfected 24 hours thereafter.

FIG. 7 shows the assay results of responses when the gene encoding a GPCR was first transfected and the gene encoding a chimeric Gqα subunit was then transfected 0 to 42 hours thereafter.

FIG. 8 shows the assay results of responses in the oocyte when the amount of the gene encoding a chimeric Gqα subunit was equivalent to that of the gene encoding a GPCR, and in the oocyte when the amount of the former gene is one tenth that of the latter gene.

FIG. 9 shows the influence of the ratio of the amount of a gene encoding a chimeric Gqα subunit to that of a gene encoding a GPCR on responses.

FIG. 10 shows a case where a ligand screening service is provided in response to a client's request.

### EMBODIMENTS FOR CARRYING OUT THE INVENTION

The present invention is hereafter described in greater detail with reference to the following examples, although the present invention is not limited to these examples.

### EXAMPLE 1

H₁ and H₂ receptors, M₁ to M₅ receptors, δ opioid receptors, and mGlu1 receptors were used as GPCRs, and a G₁₁ protein and a G₁₄ protein were used as examples of G-proteins for constitution of a chimeric Gqα subunit. They were subjected to coexpression in Xenopus oocytes. The H₁ receptor gene was obtained by a cloning technique by PCR based on sequence information (SEQ ID NO: 1) described in, for example, Biochem. Biophys. Res. Commun. 201 (2), 894-901 (1994). The H₂ receptor gene was obtained by a cloning technique by PCR based on sequence information (SEQ ID NO: 2) described in, for example, FEBS Lett. 451 (3), 327-331 (1999). Other genes were obtained from Dr. Toshihide Nukada at the Institute of Clinical Psychiatry, Tokyo. Assay of Ca-dependent Cl current is employed as a method for assaying signal transduction mediated by a GPCR, although the present invention is not necessarily limited to this assay technique.

Oocytes removed from a female Xenopus were selected by a conventional method. RNA encoding the H₁ and H₂ receptors, M₁ to M₅ receptors, δ opioid receptors, or mGlu1 receptors that were synthesized in vitro were transfected into the selected oocyte together with RNA encoding a chimeric Gqα subunit constituted by a portion of the G₁₁ protein α subunit and a portion of the G₁₄ protein α subunit that was similarly synthesized. As a control, an oocyte to which RNA of the chimeric Gqα subunit was not to be transfected was prepared. Thereafter, oocytes were cultured.

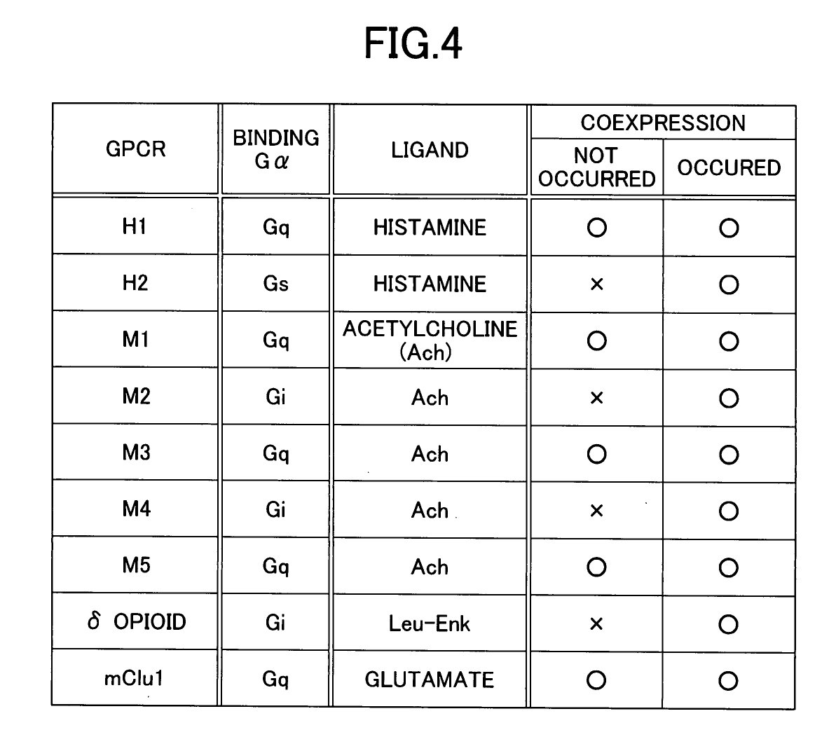

The cultured oocytes were clamped at −60 mV by TEVC. As shown in FIG. 4, a GPCR ligand, histamine, Ach, Leu-Enk, or glutamate, which was selected in dependence on the type of GPCR transfected, was added to the each transfected oocyte. The occurrence of Ca-dependent Cl response was then assayed.

The H₁, M₁, M₃, M₅, and mGlu1 GPCR receptors that naturally couple with a G-protein of the Gq subtype, could induce responses regardless of the occurrence of the coexpression of a chimeric Gqα subunit. The H₂, M₂, M₄, and δ opioid receptors that naturally couple with a G-protein of the Gs or Gi subtype could induce responses only when the chimeric Gqα subunit was coexpressed. When the G₁₁ protein or G₁₆ protein instead of the chimeric Gqα subunit was coexpressed, such effect was not obtained.

### EXAMPLE 2

The correlation between ligand concentration and response was inspected by the method described in Example 1. Three kind of coexpressed oocyte: (1) an oocyte in which the H₂ receptor and the chimeric Gqα subunit constituted by a portion of the G₁₁α subunit and a portion of the G₁₄α subunit are coexpressed, (2) an oocyte in which the M₁ receptor and the chimeric Gqα subunit constituted by a portion of the G₁₁α subunit and a portion of the G₁₄α subunit are coexpressed, and (3) an oocyte in which the M₂ receptor and the chimeric Gqα subunit constituted by a portion of the G₁₁α subunit and a portion of the G₁₄α subunit are coexpressed, were prepared in the same manner as in Example 1.

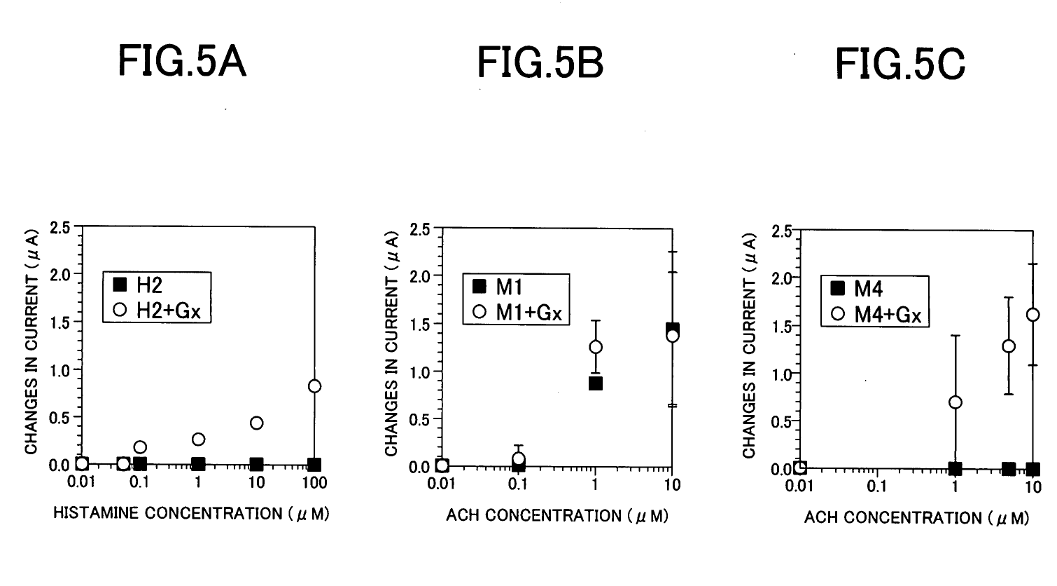

Responses generated by stimuli given by ligands (H₂: histamine, M₁/₄:Ach) at various concentration levels were measured by the method described in Example 1. Results of the measurements taken with reference to the H₂, M₁, and M₄ receptors are shown in FIG. 5. This demonstrated that there were correlations between ligand concentration and size of response when the chimeric Gqα subunit was coexpressed. Concerning the cell (2) expressing M₁ receptor that naturally couples with the G-protein of the Gq subtype, there was no significant difference in responses generated by the coexpression of the chimeric Gqα subunit. It was also confirmed that the expression of the foreign G-protein did not affect responsiveness.

### EXAMPLE 3

Whether the timing of gene transfection would affect response or not was examined using the H₂ GPCR receptor and the chimeric Gqα subunit constituted by a portion of the G₁₁α subunit and a portion of the G₁₄α subunit to be transfected.

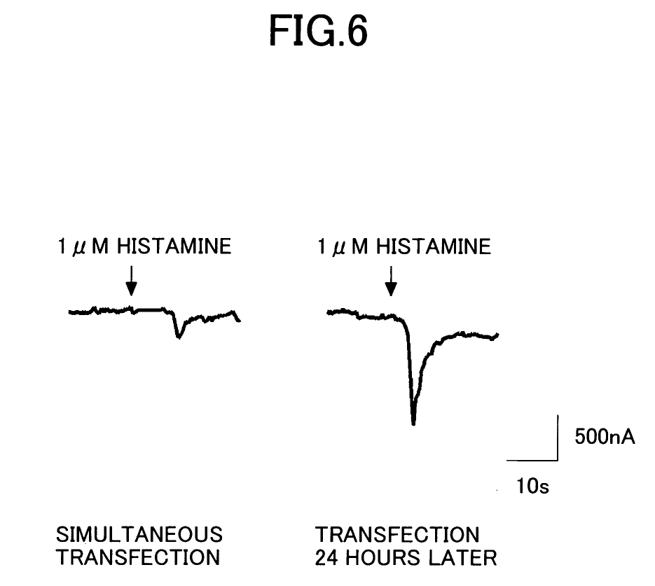

RNA encoding the H₂ receptor was transfected in the oocytes. Simultaneously or 24 hours thereafter, RNA encoding the chimeric Gqα subunit constituted by a portion of the G₁₁α subunit and a portion of the G₁₄α subunit was transfected therein. Thereafter, the ligand responses of these oocytes were assayed by the method described in Example 1. Histamine (10 μM) was used as a ligand. As shown in FIG. 6, when the gene encoding a GPCR is first transfected and the gene encoding the chimeric Gqα subunit is transfected 24 hours thereafter, responses generated by histamine were greatly increased compared with the case when both genes had been simultaneously transfected.

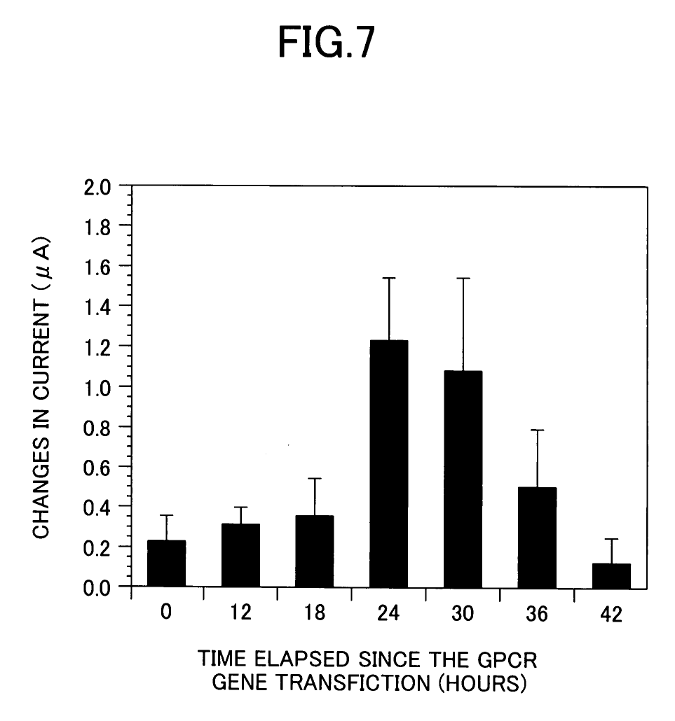

FIG. 7 shows the assay results of responses when the gene encoding GPCR is first transfected and the gene encoding the chimeric Gqα subunit is then transfected 0 to 42 hours thereafter (0 hours after the transfection refers to simultaneous transfection). As seen from FIG. 7, preferably, the gene encoding the GPCR is first transfected and the gene encoding the chimeric Gqα subunit is then transfected 12 to 36 hours thereafter.

### EXAMPLE 4

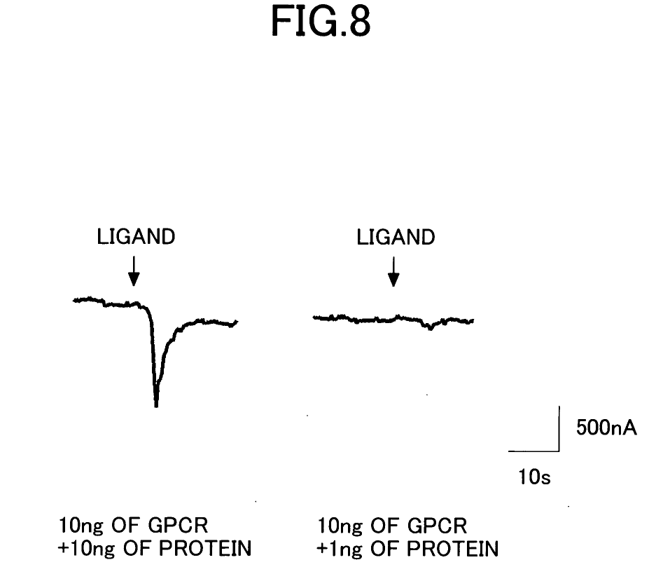

RNAs encoding a GPCR were transfected to the oocytes in amounts of 10 ng per oocyte. 24 hours thereafter, RNAs encoding the chimeric Gqα subunit constituted by a portion of the G₁₁α subunit and a portion of the G₁₄α subunit had been transfected therein in amounts of 1 ng or 10 ng per oocyte. Thereafter, the ligand responses of these oocytes were assayed by the method described in Example 1. As shown in FIG. 8, a response was observed when the amount of RNA encoding the chimeric Gqα subunit was one tenth that of RNA encoding the GPCR. A response to the ligand was apparently greater when the amount of RNA encoding the chimeric Gqα subunit transfected was the same as that of RNA encoding the GPCR, i.e., 10 ng.

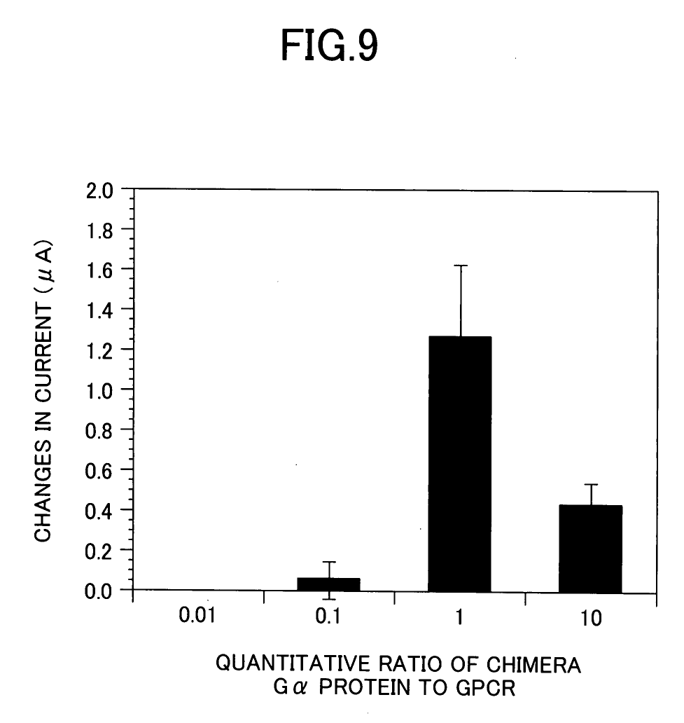

FIG. 9 shows the assay results of responses when the ratio of the amount of the gene encoding the chimeric Gqα subunit to that of the gene encoding the GPCR was 1:0.1 to 1:10. As shown in FIG. 9, the ratio of the amount of the gene encoding the chimeric Gqα subunit to that of the gene encoding the GPCR is preferably 1:0.1 to 1:10.

### EXAMPLE 5

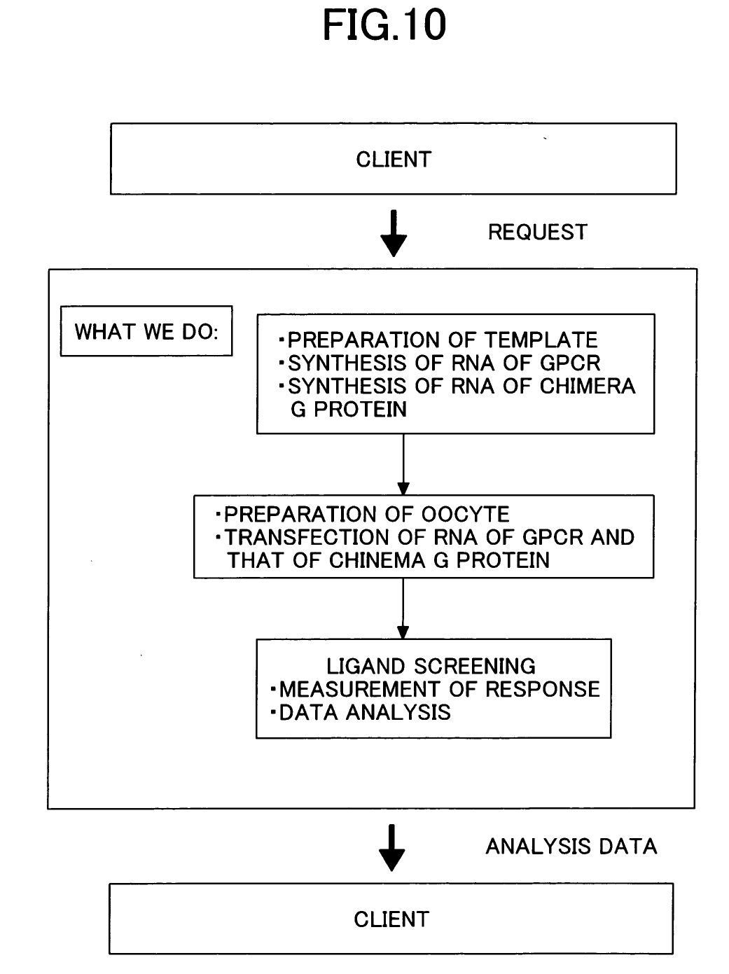

An embodiment of the present invention is hereafter described in detail with reference to FIG. 10.

RNA encoding a GPCR and RNA encoding the chimeric Gα subunit are synthesized. DNA encoding a GPCR that was provided from a client is processed with an enzyme, and a template is prepared. Simultaneously, DNA encoding the chimeric Gqα subunit is cleaved with an enzyme, and a template is prepared. Based thereon, RNA encoding the GPCR and RNA encoding the chimeric Gqα subunit are transfected together in the Xenopus oocytes. Culture is conducted for a given period of time, candidate substances of GPCR ligands are added to the oocytes, and cellular response thereupon is detected. Thus, GPCR ligands are screened.

Effect of the Invention

The present invention enables assay of the activity of signal transduction systems mediated by GPCRs by employing PI turnover activity as an indicator in a foreign protein-expressing system that utilizes animal oocytes such as Xenopus oocytes, regardless of the G-protein subtype to which the GPCR would naturally couple.

1. A method for preparing foreign protein-expressing cells, wherein genes encoding G-protein coupled receptors (GPCRs) and genes encoding a chimeric Gqα subunit constituted by a portion of a Gqα or G₁₁α subunit and a portion of a G₁₄α, G₁₅α, or G₁₆α subunit are transfected into animal cells and expressed therein.

2. The method for preparing foreign protein-expressing cells according to claim 1, wherein the amino acid sequence of the N-terminal side of the chimeric Gqα subunit is derived from a Gq or G₁₁ subunit and the amino acid sequence of the C-terminal side thereof is derived from a G₁₄, G₁₅, or G₁₆ subunit.

3. The method for preparing foreign protein-expressing cells according to claim 1, wherein a gene encoding a GPCR is first transfected and a gene encoding the chimeric Gqα subunit is then transfected 12 to 36 hours thereafter.

4. The method for preparing foreign protein-expressing cells according to claim 1, wherein the ratio of the amount of genes encoding the chimeric Gqα subunit to that of the genes encoding a GPCR is 1:0.1 to 1:10.

5. A group of foreign protein-expressing cells comprising a G-protein coupled receptor (GPCR) and a chimeric Gqα subunit constituted by a portion of a Gqα or G₁₁α subunit and a portion of a G₁₄α, G₁₅α, or G₁₆α subunit.

6. The group of foreign protein-expressing cells according to claim 5, wherein the amino acid sequence of the N-terminal side of the chimeric Gqα subunit is derived from a Gq or G₁₁ subunit and the amino acid sequence of the C-terminal side thereof is derived from a G₁₄, G₁₅, or G₁₆ subunit.

7. A screening method, wherein a test substance is brought into contact with foreign protein-expressing cells comprising a G-protein coupled receptor (GPCR) and a chimeric Gqα subunit constituted by a portion of a Gqα or G₁₁α subunit and a portion of a G₁₄α, G₁₅α, or G₁₆α subunit, GPCR activities are assayed, and a ligand of the GPCR is then screened for.

8. The screening method according to claim 7, wherein elevation of intracellular Ca concentration is assayed.

9. The screening method according to claim 7, wherein changes in a Ca-dependent Cl current are assayed as indicators of intracellular Ca concentration.

10. The screening method according to claim 7 to 9, wherein the amino acid sequence of the N-terminal side of the chimeric Gqα subunit is derived from a Gq or G₁₁ subunit and the amino acid sequence of the C-terminal side thereof is derived from a G₁₄, G₁₅, or G₁₆ subunit.

11. The screening method according to claim 8, wherein the amino acid sequence of the N-terminal side of the chimeric Gqα subunit is derived from a Gq or G₁₁ subunit and the amino acid sequence of the C-terminal side thereof is derived from a G₁₄, G₁₅, or G₁₆ subunit.

12. The screening method according to claim 9, wherein the amino acid sequence of the N-terminal side of the chimeric Gqα subunit is derived from a Gq or G₁₁ subunit and the amino acid sequence of the C-terminal side thereof is derived from a G₁₄, G₁₅, or G₁₆ subunit.
